(19) United States
(12) Patent Application Publication
(10) Pub. No.: US 2005/0043663 A1
(43) Pub. Date: Feb. 24, 2005

(54) Folding bed with a spine-stretching function

(76) Inventor: Cheng-Hsien Lin, Tainan Hsien (TW)

Correspondence Address: CHARLES E. BAXLEY, ESQUIRE, Third Floor, 90 John Street, New York, NY, 10038, US

(21) Appl. No.: 10/642,792

(22) Filed: Aug. 18, 2003

(51) Int. Cl. A61F 5/00

(52) U.S. Cl. 602/33

(57) ABSTRACT

A bed includes a front frame having a front mattress mounted thereon, a rear frame having a rear mattress mounted thereon, a stretching mechanism and a legrest device. The stretching mechanism includes two handles mounted in front of the front frame for a user's gripping. The handles are movable along a longitudinal direction of the bed. The legrest device is mounted to the rear frame and movable relative to the rear frame along the longitudinal direction of the bed. The legrest positions the feet of the user. A first elastic element is attached between the rear frame and the legrest device. When a stretching force provided by the stretching mechanism imparted to the legrest device is greater than a pulling stress of the first elastic element, the first elastic element is pulled and thus moves the legrest device along the longitudinal direction relative to the rear frame.

### BACKGROUND OF THE INVENTION

1. Field of the Invention

The present invention relates to a folding bed. In particular, the present invention relates to a bed with a spine-stretching function.

2. Description of the Related Art

The spine of a human body often bends due to long-term sitting, improper standing gestures, lacking exercising, or aging. A typical bed merely provides a lying function without stretching the human body. FIG. 10 of the drawings illustrates a conventional bed with a spinal correction device including a frame 1′, a plate 2′, and a legrest 3′. In use, a user lies on the plate 2′with his or her legs being hooked by the legrest 3′. The plate 2′is turned to move the user's to an upside-down position, thereby stretching the spine of the user by gravity.

However, the user must keep the upside-down position for a long time and thus causes discomfort, as blood flows toward the user's head. Further, the user might be injured if the legrest 3′fail to provide a reliable hooking function. Further, the plate 2′could not be located in a horizontal position and thus fail to provide the basic function of lying for the user.

### SUMMARY OF THE INVENTION

An object of the present invention is to provide a folding bed that may stretch the user's spine while providing a lying function.

A bed in accordance with the present invention comprises:
a front frame, at least one front leg being attached to the front frame;
a rear frame, at least one rear leg being attached to the rear frame;
a front mattress mounted on the front frame;
a rear mattress mounted on the rear frame;
a stretching mechanism including two handles mounted in front of the front frame for a user's gripping, the handles being movable along a longitudinal direction of the bed; and
a legrest device mounted to the rear frame and movable relative to the rear frame along the longitudinal direction of the bed, the legrest being adapted to position feet of the user, a first elastic element having a first end attached to the rear frame and a second end attached to the legrest device;
wherein when a stretching force provided by the stretching mechanism imparted to the legrest device is greater than a pulling stress of the first elastic element, the first elastic element is pulled and thus moves the legrest device along the longitudinal direction relative to the rear frame, and a returning force of the first elastic element buffering the stretching movement.

Other objects, advantages, and novel features of the invention will become more apparent from the following detailed description when taken in conjunction with the accompanying drawings.

### BRIEF DESCRIPTION OF THE DRAWINGS

FIG. 1 is an exploded perspective view of a folding bed in accordance with the present invention.

FIG. 2 is a perspective view of the folding bed in accordance with the present invention, wherein a front mattress and a rear mattress are removed for clarity.

FIG. 3 is a side view of the folding bed in accordance with the present invention.

FIG. 4 is a top view of the folding bed in accordance with the present invention.

FIG. 5 is a view similar to FIG. 3, illustrating use of the folding bed in accordance with the present invention.

FIG. 6 is a top view of the folding bed under the stretching operation.

FIG. 7 is a side view of the folding bed, illustrating another use of the folding bed.

FIG. 8 is a view illustrating a legrest device of the folding bed in a storage position.

FIG. 9 is a side view of the folding bed in a folded state.

FIG. 10 is a schematic side view of a conventional bed with a spine-correcting function.

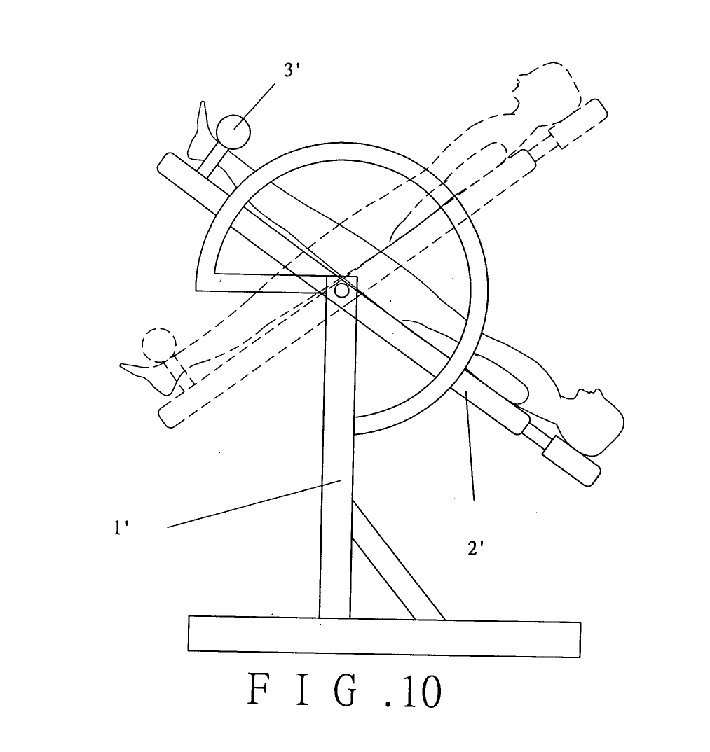

### DETAILED DESCRIPTION OF THE PROFFERED EMBODIMENT

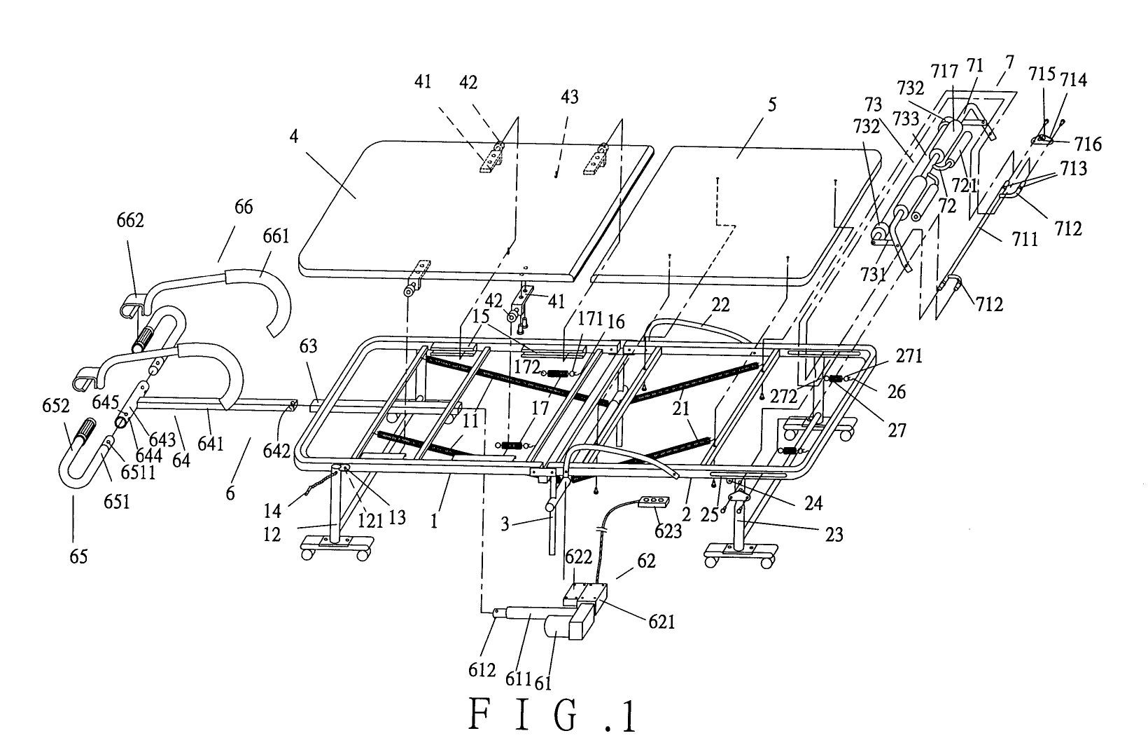

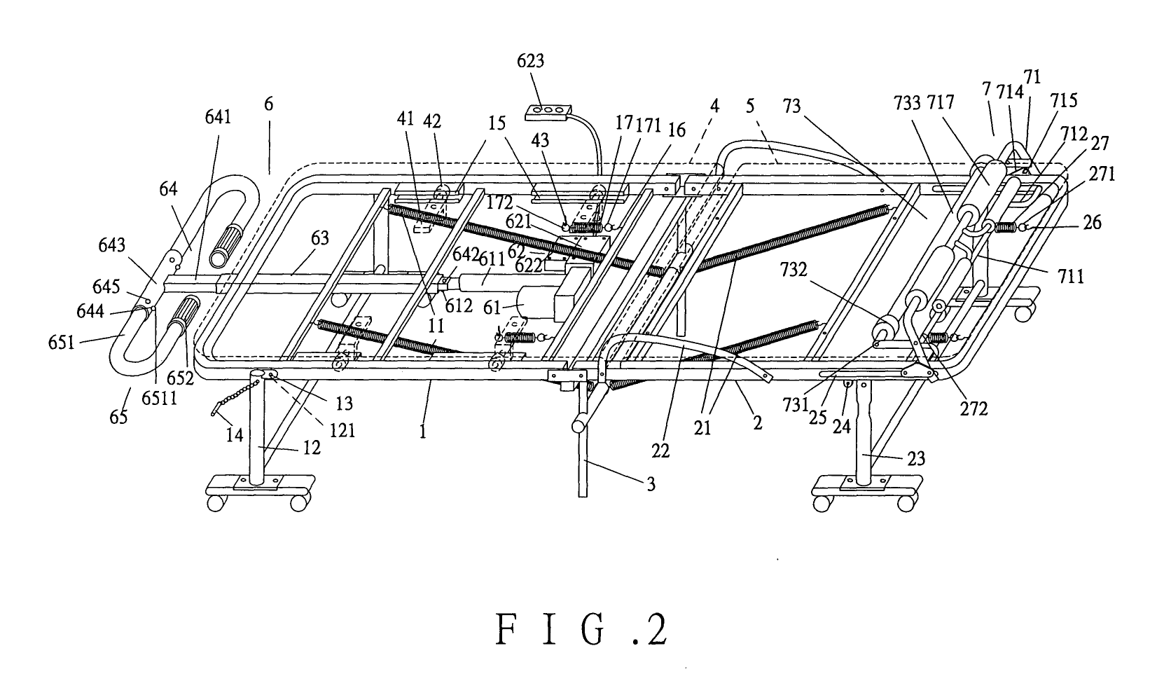

Referring to FIGS. 1 and 2, a folding bed in accordance with the present invention generally comprises a front frame 1, a rear frame 2, a front mattress 4, a rear mattress 5, a stretching mechanism 6, and a legrest device 7. The front frame 1 has an end pivotally connected to an intermediate frame 3 between the front frame 1 and the rear frame 2. At least one elastic element 11 (two in this embodiment) has a front end attached to the front frame 1 and a rear end attached to the intermediate frame 3. A front leg 12 is mounted to each of two lateral sides of the front frame 1. A pin 14 is inserted into a positioning hole 13 in the respective lateral side of the front frame 1 and a positioning hole 121 in the respective front leg 12, thereby securing the respective front leg 12 to the front frame 1. A guide track 15 is provided on an inner surface of the respective lateral side of the frame 1, allowing sliding movement of the front mattress 4, which will be described later. An end 171 of at least one elastic element 17 (two in this embodiment) is attached to a connecting portion 16 provided on a rear end of the front frame 1.

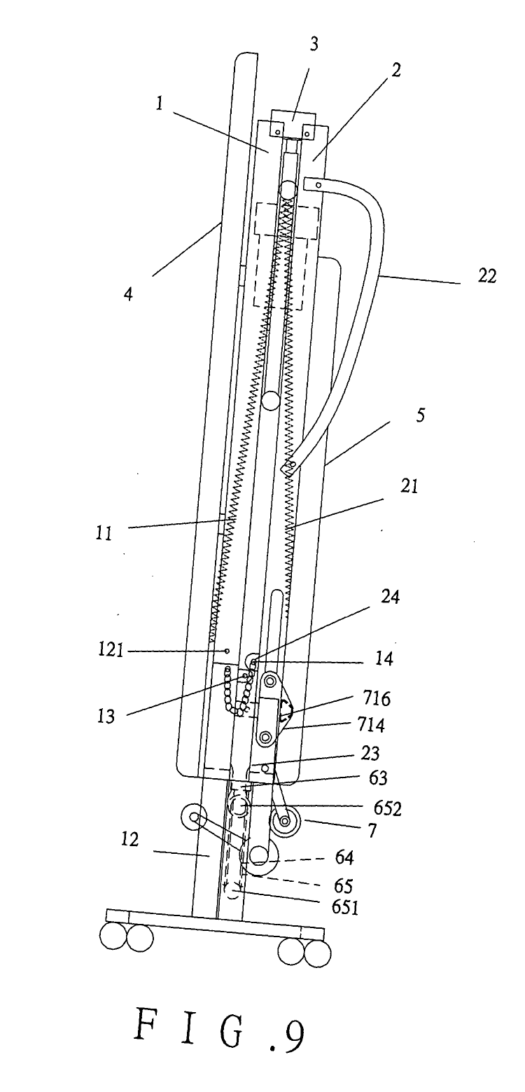

The rear frame 2 includes an end pivotally connected to the intermediate frame 3. At least one elastic element 21 (two in this embodiment) has a front end attached to the intermediate frame 3 and a rear end attached to the rear frame 2. A handle 22 is provided on each of two lateral sides of the rear frame 2 for easy carriage when the folding bed is in a folded state (FIG. 9). A rear leg 23 is mounted to each lateral side of the rear frame 2. Further, the rear frame 2 has a positioning hole 24 and an elongated guide groove 25 in an inner surface of each lateral side thereof. Further, an end 271 of at least one elastic element 27 (two in this embodiment) with a proper resiliency is attached to a connecting portion 26 on a rear end of the rear frame 2.

The front mattress 4 is longer than the front frame 1 and has at least one wheel seat 41 mounted to each of two lateral sides thereof. A wheel 42 is rotatably mounted to the wheel seat 41 and rollably received in the respective track 15 of the front frame 1. Thus, the front mattress 4 may slide along a longitudinal direction of the bed. Further, the other end 172 of the respective elastic element 17 is attached to a connecting portion 43 on a rear portion of the front mattress. The rear mattress 5 is mounted on the rear frame 2.

The stretching mechanism 6 includes a motor 61, a controller 62, a guide member 63, a connecting member 64, two handles 65, and two hook members 66. The motor 61 is mounted below the front frame 1 and includes a telescopic rod 611 that can be driven by the motor 61 to move to and from. The telescopic rod 611 has a spring-biased button 612 on a distal end thereof. The motor 61 is controlled by the controller 62 that includes a transformer 621, a control circuit 622, and a manual control 623. The motor 61 is controlled by the manual control 623 to move the telescopic rod 611 to and from within a predetermined travel. The guide member 63 is fixed to an underside of the front frame 1 and extends along a longitudinal direction of the bed. The connecting member 64 includes a main section 641 slidably received in the guide member 63, with a hole 642 in an end of the main section 641 being engaged with the spring-biased button 612 of the telescopic rod 611. Thus, the connecting member 64 slides along the longitudinal direction of the bed when the motor 61 is driven.

A coupling tube 643 is provided on the other end of the connecting member 64 and extends in a direction orthogonal to an extending direction of the main section 641. The coupling tube 643 includes two ends each having a vertical positioning hole 645 and a horizontal positioning hole 644. Each handle 65 includes a connecting portion 651 on an end thereof and a bent grip portion 652 on the other end thereof. The connecting portion 651 includes a spring-biased button 6511 engaged with one of the positioning holes 644 and 645 on the respective end of the coupling tube 643. Each hook member 66 includes a hook portion 661 on an end thereof for engaging with an armpit of a user. An engaging member 662 is provided on the other end of each hook member 66 for engaging with the grip portion 652 of the respective handle 65.

The legrest device 7 includes a movable frame 71, two footrests 72, and a support rod 73. The movable frame 71 is substantially U-shaped and mounted to the rear end of the rear frame 2. A connecting rod 711 includes two ends each having an extension 712 for engaging with the respective end of the movable frame 71. Each end of the connecting rod 711 extends through the respective lateral side of the rear frame 2 and has a wheel 713 rotatably mounted thereon, the wheel 713 being rollably received in the respective guide groove 25 of the rear frame 2. Similarly, each end of the extension 712 extends through the respective lateral side of the rear frame 2 and has a wheel 713 rotatably mounted thereon, the wheel 713 being rollably received in the respective guide groove 25 of the rear frame 2. Further, a positioning plate 714 is attached to the respective end of the connecting rod 711 and the respective end of the respective extension 712. Each positioning plate 714 includes a first abutting portion 715 for the folding bed in an unfolded state and a second abutting portion 716 for the folding bed in the folded state. The other end 272 of the respective elastic element 27 is attached to the connecting rod 711.

Further, two instep pads 717 are provided on the movable frame 71 for providing a comfortable contact with the insteps of the user. The respective footrest 72 is located behind the respective instep pad 711 on the movable frame 71 and has a sole pad 721 mounted thereon for providing a comfortable support for the sole of the user. The support rod 73 is located in front of the movable frame 71 and has two ends respectively pivotally connected to the ends of the movable frame 71. A wheel 732 is rotatably mounted to each end of the support rod 73 so as to roll on the rear mattress 5. A heel pad 733 is provided on a central portion of the support rod 73 for providing a comfort support for the heels of the user.

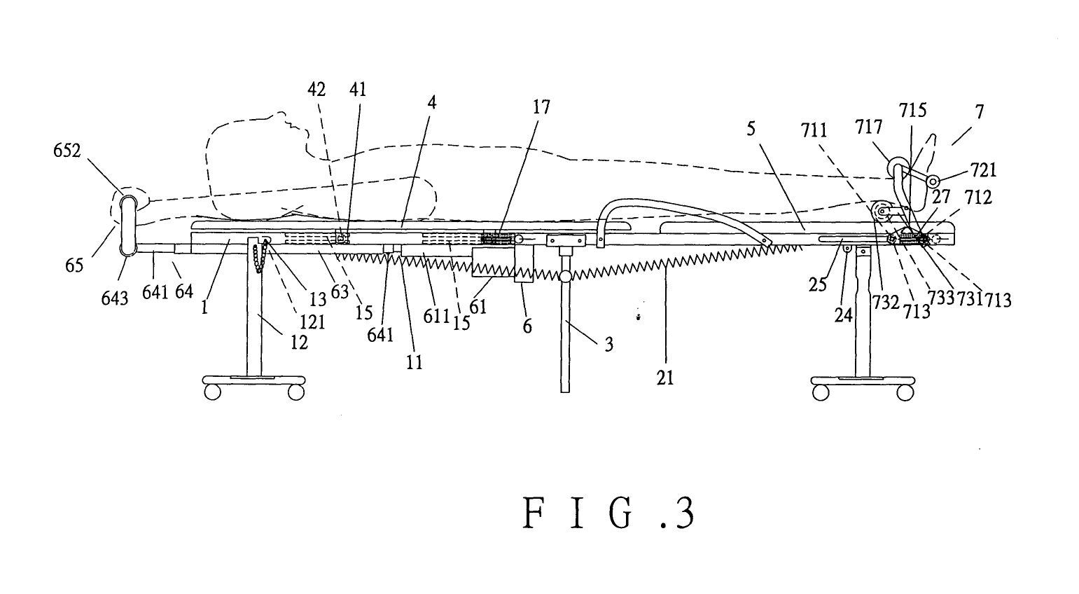

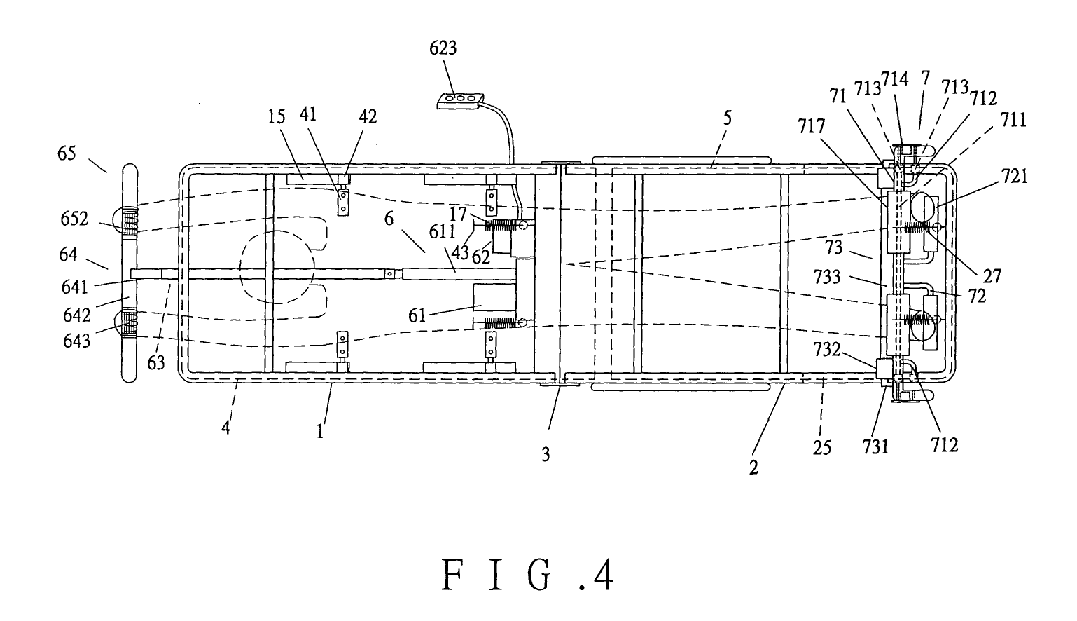

Still referring to FIGS. 1 and 2 and further to FIGS. 3 and 4, in use, the spring-biased button 6511 on the respective handle 65 is engaged in, e.g., the respective vertical positioning hole 645 of the connecting tube 643. The user lies on the bed, with his or her insteps abutting against the instep pads 717, with his or her sole resting on the sole pads 721, with his or her heels resting on the heel pads 733, with his or her center of gravity being located on the front mattress 4.

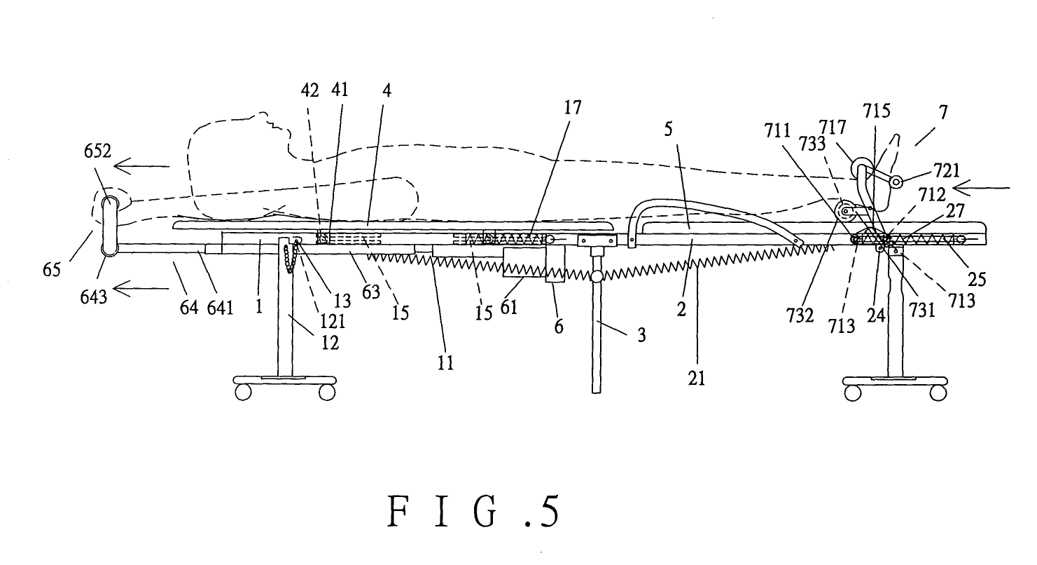

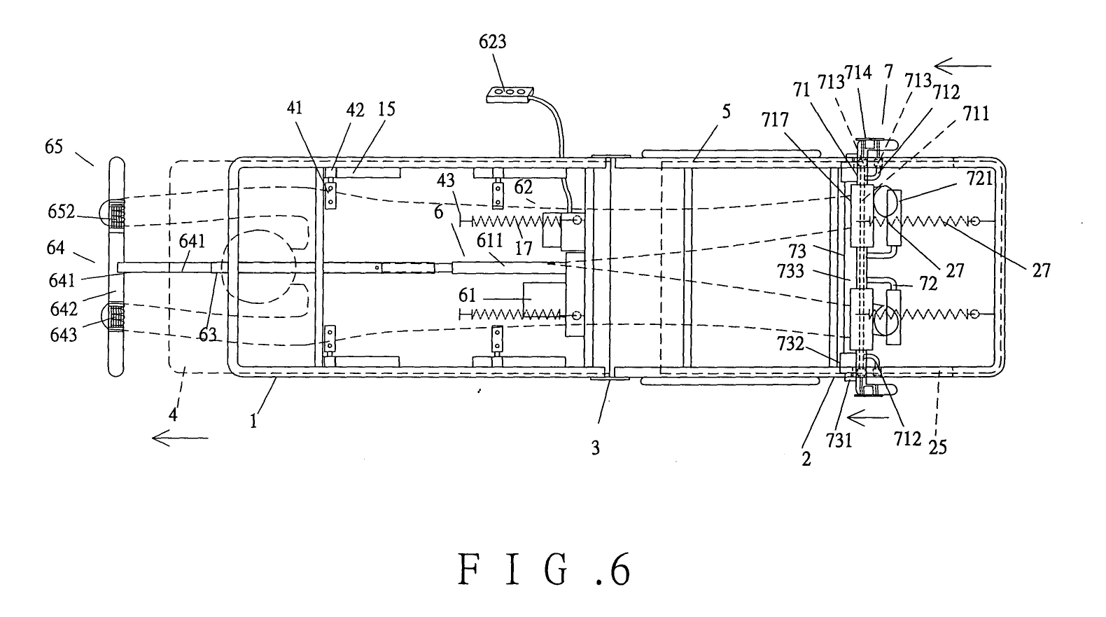

After inputting predetermined travel and predetermined time into the manual control 623, the user grips the handles 65. Referring to FIGS. 5 and 6, the motor 61 is activated at the determined time, moving the connecting member 64 forward to the determined travel and thus stretching the spine of the user. The front mattress 4 slides forward while the wheels 42 are sliding along the tracks 15. The elastic elements 17 are stretched to avoid friction between the human body and the front mattress 4. The front mattress 4 returns to its original position under the action of the elastic elements 17 when desired.

The movable frame 71 is subjected to a pulling force greater than the pulling stress of the elastic elements 27. The elastic elements 27 are thus pulled to an appropriate extent. The wheels 713 on the connecting rod 711 and the extensions 712 roll along the guide grooves 25 of the rear frame 2, providing smooth sliding movement and buffering the stretching movement to prevent excessive stretching of the spine of the user. Further, when the support rod 73 moves forward, damage to the rear mattress 5 is prevented by means of rolling movement of the wheels 732 on the rear mattress 5. The user's spine is thus stretched to an appropriate extent to correct the spine and to restore energy.

The controller 62 activates the motor 61 to turn in a reverse direction when the handles 65 reach a predetermined position in the forward travel. The handles 65 are moved rearward, and the front mattress 4 and the legrest device 7 return their original position under the action of the elastic elements 17 and 27. The stretching movement can be repeated as many times as desired.

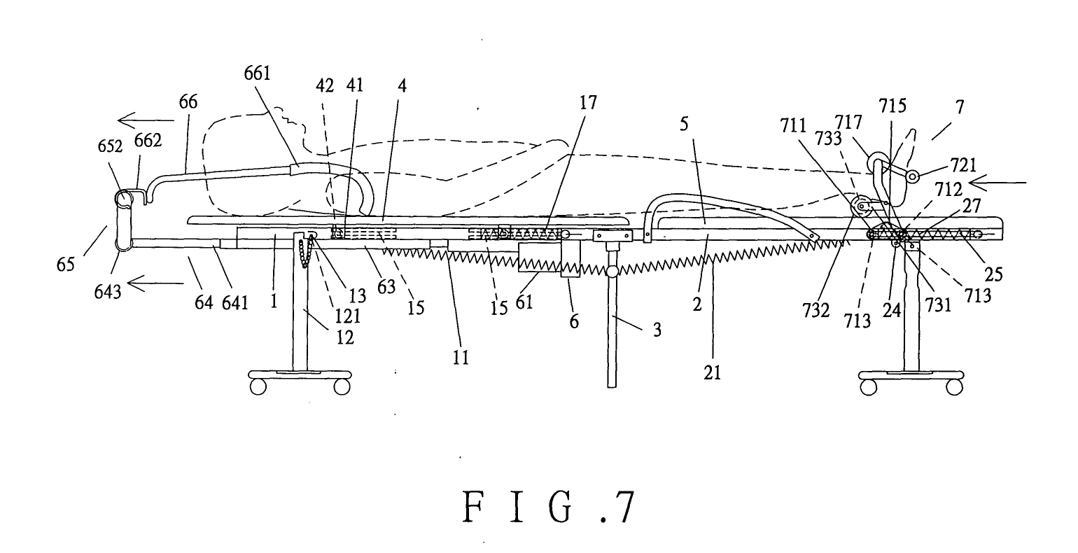

Referring to FIG. 7, the hook members 66 can be attached to the handles 65 by means of engaging the engaging members 662 with the handles 65. The hook portion 661 of the respective hook member 66 provides a support for the respective armpit of the user. In this case, the user needs not to grip the handles 65.

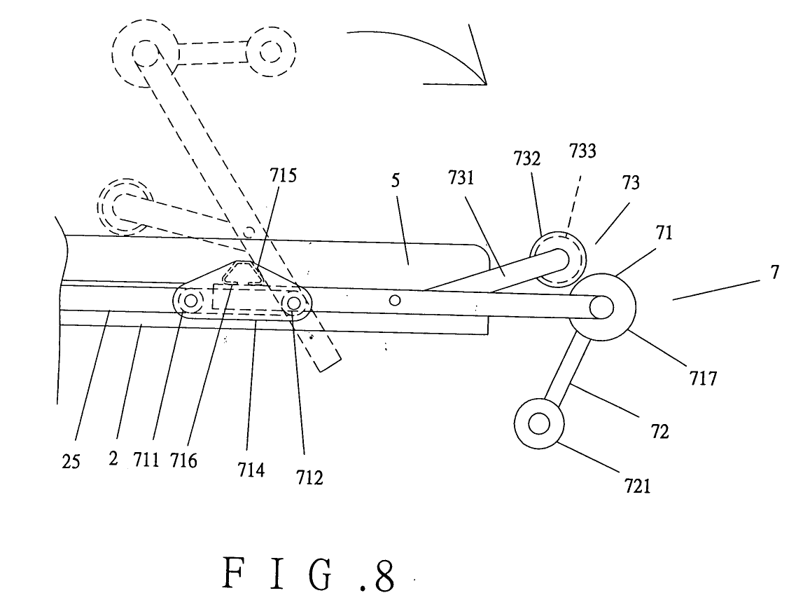

Referring to FIGS. 8 and 9, when not in use, the spring-biased button 651 of the respective handle 65 is disengaged from the respective vertical positioning hole 645 and then engaged in the respective horizontal positioning hole 644 of the coupling tube 643. Further, the legrest 7 pivots about the extensions 712 of the connecting rod 711 to a position shown by the solid lines in FIG. 8 for engaging with the second abutting section 716. The legrest 7 is thus retained in place, reducing the space for storage when the folding bed is in the folded state. For folding the bed, the intermediate frame 3 is moved upward, and the front bed frame 1 and the rear bed frame 2 are moved to the folded positions under the action of the elastic elements 11 and 21. The pin 14 is inserted into the positioning hole 24 of the rear bed frame 2, thereby fixing the folding bed in the folded state, best shown in FIG. 9.

In conclusion, the folding bed in accordance with the present invention includes the following advantages:
1. The user may lie flat on the bed, which is suitable for all ages while providing improved safety.
2. The whole body of the user can be stretched and the user feels restored.
3. The spine of the user can be appropriately stretched, providing a spinal correction.
4. The legrest device is slidable during the stretching operation, preventing the user's spine from being excessively stretched.
5. The front mattress is slidable, preventing friction between the front mattress and the user's body, providing improved comfort, and preventing the front mattress from being damaged.
6. The hook members 66 allow the user not to grip the handles 65 during the stretching operation, providing improved utility.
7. The handles 66 and the legrest 7 as well as the whole folding bed can be folded to reduce the space required for storage and transport.

Although the invention has been explained in relation to its preferred embodiment, it is to be understood that many other possible modifications and variations can be made without departing from the scope of the invention as hereinafter claimed.

1. A bed comprising:
a front frame, at least one front leg being attached to the front frame;
a rear frame, at least one rear leg being attached to the rear frame;
a front mattress mounted on the front frame;
a rear mattress mounted on the rear frame;
a stretching mechanism including two handles mounted in front of the front frame for a user's gripping, the handles being movable along a longitudinal direction of the bed; and
a legrest device mounted to the rear frame and movable relative to the rear frame along the longitudinal direction of the bed, the legrest being adapted to position feet of the user, a first elastic element having a first end attached to the rear frame and a second end attached to the legrest device;
wherein when a stretching force provided by the stretching mechanism imparted to the legrest device is greater than a pulling stress of the first elastic element, the first elastic element is pulled and thus moves the legrest device along the longitudinal direction relative to the rear frame, and a returning force of the first elastic element buffering the stretching movement.

2. The bed as claimed in claim 1, wherein the front frame includes two lateral sides each having a track defined therein, a second elastic element having a first end attached to the front frame and a second end attached to the front mattress, the front mattress including two lateral sides each having a wheel seat attached thereto, a wheel being rotatably mounted to the respective wheel seat and rollably received in the respective track of the front frame.

3. The bed as claimed in claim 1, wherein the stretching device further includes a motor, a controller, a guide member, and a connecting member, the motor being mounted below the front frame and including a telescopic rod that can be driven by the motor to move to and from, the guide member extending in the longitudinal direction of the bed, the connecting member including a main section slidably guided by the guide member, the main section having an end engaged with the telescopic rod to move therewith, the connecting member further including a coupling tube on another end thereof, the coupling tube including two ends, the respective handle including a connecting portion on a first end thereof and a bent grip portion on a second end thereof, the connecting portion of the respective handle being engaged with the respective end of the coupling tube.

4. The bed as claimed in claim 3, wherein the main section of the connecting member is slidably received in the guide member.

5. The bed as claimed in claim 3, wherein the end of the main section of the connecting member includes a hole, the telescopic rod having a distal end with a spring-biased button engaged with the hole of the main section of the connecting member.

6. The bed as claimed in claim 3, wherein the connecting portion of the respective handle includes a spring-biased button, the respective end of the coupling tube having a horizontal positioning hole and a vertical positioning hole one of which is engaged with the spring-biased button of the connecting portion of the respective handle.

7. The bed as claimed in claim 1, wherein the stretching mechanism further includes two hook members each having a first end releasably engaged with the grip portion of the respective handle and a second end on which a hook portion is formed, the hook portion being adapted to be engaged with an armpit of the user.

8. The bed as claimed in claim 1, wherein the rear frame includes two lateral sides each having a guide groove defined therein, the legrest device including a movable frame, two footrests, and a support rod, the movable frame being substantially U-shaped and mounted to the rear end of the rear frame, a connecting rod having two ends each having an extension for engaging with a respective end of the movable frame, each said end of the connecting rod extending through the respective lateral side of the rear frame and including a first wheel rotatably mounted thereto, the extension having two ends each extending through the respective lateral side of the rear frame and having a second wheel rotatably mounted thereto, the first wheel and the second wheel being rollably received in the respective guide groove of the rear frame, the second end of the first elastic element being attached to the connecting rod, the support rod being mounted in front of the movable frame and including two ends respectively pivotally connected to the ends of the movable frame, a third wheel being rotatably mounted to each said end of the support rod and rollable on the rear mattress.

9. The bed as claimed in claim 8, wherein the movable frame includes two instep pads mounted thereon.

10. The bed as claimed in claim 8, wherein the respective footrest includes a sole pad mounted thereon.

11. The bed as claimed in claim 9, wherein the respective footrest includes a sole pad mounted thereon.

12. The bed as claimed in claim 8, wherein the support rod includes a heel pad mounted thereon.

13. The bed as claimed in claim 9, wherein the support rod includes a heel pad mounted thereon.

14. The bed as claimed in claim 11, wherein the support rod includes a heel pad mounted thereon.

15. The bed as claimed in claim 1, further including an intermediate frame mounted between the front frame and the rear frame, the front frame including an end pivotally connected to the intermediate frame, the rear frame including an end pivotally connected to the intermediate frame.
